(19) United States
(12) Patent Application Publication
(10) Pub. No.: US 2013/0109989 A1
(43) Pub. Date: May 2, 2013

(54) METHOD AND APPARATUS FOR OBTAINING AND PROCESSING BALLISTOCARDIOGRAPH DATA

(71) Applicants: Edward Busse, Qualicum Beach (CA); Graeme Jahns, Burnaby (CA); James Alexander Burns, Vancouver (CA)

(72) Inventors: Edward Busse, Qualicum Beach (CA); Graeme Jahns, Burnaby (CA); James Alexander Burns, Vancouver (CA)

(73) Assignee: HEART FORCE MEDICAL INC.

(21) Appl. No.: 13/378,610

(22) PCT Filed: Jun. 17, 2010
(86) PCT No.: PCT/CA10/00895

§ 371 Date: Jul. 5, 2012

Related U.S. Application Data
(60) Provisional application No. 61/218,025, filed on Jun. 17, 2009.

(51) Int. Cl. A61B 5/11; A61B 5/02; A61B 5/029; A61B 5/00

(52) U.S. Cl. CPC A61B 5/1102; A61B 5/7278; A61B 5/02028; A61B 5/7282; A61B 5/029
USPC 600/527

(57) ABSTRACT

A method and apparatus are provided for obtaining and processing ballistocardiograph data to determine a physiological condition of a subject. Ballistocardiograph data indicative of heart motion of the subject measured along a plurality of spatial axes by a sensor device which may comprise a three-axis accelerometer. The ballistocardiograph data is processed to determine processed data indicative of heart motion of the subject. Indications of physiological condition are determined based at least in part on the processed data. Processing may comprise aggregation of multidimensional data, determining magnitude of heart motion and derivative thereof, determining a thrust summation, determining an index value, outputting a report based on an index value, etc. Processing may be informed by operator input, such as a time window of interest or indications of interest.

### FIELD OF THE INVENTION

The present invention pertains in general to technology for determining a physiological condition of a subject and in particular to a method and apparatus for obtaining and processing ballistocardiograph data.

### BACKGROUND

Cardiovascular disease is one of the leading causes of death in the Western world, with incidences expected to increase over the coming years as the “baby boomer” generation ages. As a result, technologies that improve or aid the assessment and/or detection of cardiovascular disease will become increasingly important in patient monitoring and overall care.

Currently, devices such as electrocardiogram (ECG) monitors and echocardiograms are used in the identification and assessment of cardiovascular disease. The ECG provides a fairly rapid electrical assessment of the heart, but does not provide any information relating to the force of contraction. Echocardiography provides images of sections of the heart and can provide comprehensive information about the structure and function of the heart, but requires expensive equipment and specialised operating personnel.

Ballistocardiography involves measuring the movement caused by the percussive effects of the heart. Historically, ballistocardiograph (BCG) data was obtained while a subject lay supine on a bed that contained either an apparatus that would allow for measurement of these movements or a facilitating apparatus that was attached across the shin area of the legs. More recently, however, BCG data has been obtained using small sensor devices, such as accelerometers, which record minute movements on an individual's body surface which are representative of the movement of the heart.

U.S. patent application Ser. No. 11/895,040 (Publication No. 2009/0054742) describes an apparatus for signal detection, processing and communication that comprises two or more flexible layers, which include two or more sensors. Several examples of sensors are described, including ECG monitors or accelerometers.

U.S. patent application Ser. No. 12/254,468 (Publication No. 2009/0105601) describes a heart-rate variability analysis method and device. The device comprises a first and second cardiac action potential measuring means, which are attached to the thoracic and diaphragmatic regions, and a three-axis acceleration measuring means. The device is used to measure the R-R interval heart-rate and can exclude the effect of respiratory movement on heart-rate variability.

International Patent Application No. PCT/CA2008/000274 (Publication No. WO2008/095318) describes a system for monitoring and detecting abnormalities in an individual's physiological condition by concurrently detecting and processing an ECG and BCG signal.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method and apparatus for obtaining and processing ballistocardiograph data. In accordance with an aspect of the present invention, there is provided an apparatus for determining information indicative of a subject's physiological condition, said apparatus comprising: a sensor device configured to obtain ballistocardiograph data indicative of heart motion of the subject measured along a plurality of spatial axes; and a computing device communicatively coupled to the sensor device and configured to receive the ballistocardiograph data therefrom, the computing device configured to determine, based on the ballistocardiograph data, processed data indicative of heart motion of the subject, the computing device further configured to determine one or more indications of the subject's physiological condition based at least in part on the processed data.

In accordance with another aspect of the present invention, there is provided a method for determining information indicative of a subject's physiological condition, said method comprising: obtaining ballistocardiograph data indicative of heart motion of the subject measured along a plurality of spatial axes; determining, based on the ballistocardiograph data, processed data indicative of heart motion of the subject; and determining one or more indications of the subject's physiological condition based at least in part on the processed data.

In accordance with another aspect of the present invention, there is provided a computer program product comprising a memory having computer readable code embodied therein, for execution by a CPU, for performing a method for determining information indicative of a subject's physiological condition, said method comprising: obtaining ballistocardiograph data indicative of heart motion of the subject measured along a plurality of spatial axes; determining, based on the ballistocardiograph data, processed data indicative of heart motion of the subject; and determining one or more indications of the subject's physiological condition based at least in part on the processed data.

### BRIEF DESCRIPTION OF THE FIGURES

These and other features of the invention will become more apparent in the following detailed description in which reference is made to the appended drawings.

FIG. 1(a) is an example of an electrocardiogram waveform.

FIG. 1(b) is an example of a ballistocardiogram waveform.

FIG. 2 is a schematic diagram of an apparatus for acquiring and analyzing data relating to a physiological condition of a subject, in accordance with embodiments of the present invention.

FIG. 3 is a block diagram of an apparatus in accordance with embodiments of the present invention.

FIG. 4(a) is a block diagram of selected components of a sensor device of FIG. 2, in accordance with embodiments of the present invention.

FIG. 4(b) is a block diagram of selected components of a sensor device of FIG. 2, in accordance with embodiments of the present invention.

FIG. 5 illustrates a method for determining information indicative of a subject's physiological condition, in accordance with embodiments of the present invention.

FIG. 6 is an example of a synchronized electrocardiogram and ballistocardiogram waveform pair captured using an apparatus for acquiring and analyzing data relating to a physiological condition of a subject, in accordance with embodiments of the present invention.

FIG. 7 is a flowchart depicting a method of generating a waveform according to an embodiment of the invention.

FIG. 8a is a waveform generated using the method of FIG. 6.

FIG. 8b is an electrocardiogram synchronized with the waveform of FIG. 7a.

FIG. 9 is a flowchart depicting a method for analyzing a derivative magnitude waveform according to an embodiment of the invention.

FIG. 10 is a graph depicting an example of application of the method of FIG. 8.

FIG. 11 is an example of a derivative magnitude waveform including annotations for determining a Systolic Thrust Index (STI).

FIG. 12 is an example of a derivative magnitude waveform including annotations for determining a Systolic Thrust Window (STW).

FIG. 13 is an example of a derivative magnitude waveform including annotations for determining a Recoil Index (RI).

FIG. 14 is an example of a derivative magnitude waveform including annotations for determining a Recoil Window (RW).

FIG. 15 is an example of a derivative magnitude waveform including annotations for determining a Diastolic Ratio (DR).

FIG. 16 is a flowchart depicting a method of determining an index relating to a physiological condition of a subject, in accordance with embodiments of the present invention.

### DETAILED DESCRIPTION OF THE INVENTION

### Definitions

As used herein, the term “ballistocardiograph data” refers to a data obtained from a device for detecting and conveying motion, such as vibrations and/or accelerations, due to heart operation. For example, the device may comprise one or more motion sensors such as accelerometers for directly or indirectly detecting vibrations or accelerations in a chest wall of a subject, for example by monitoring the chest wall or another body area which moves in a correlated manner with the heart and/or chest wall. Monitored vibrations and/or accelerations may correspond to compressive motion due to heart operation, shear motion, or the like, or a combination thereof.

The term “time series data” refers to a sequence of data values representative of one or more observed, inferred, or otherwise acquired and/or processed time-varying quantities. Each data value of time series data corresponds to an implicitly or explicitly specified time, which may be measured on a real time scale, normalized or adjusted time scale, or dimensionless scale. Time series data may correspond to data captured at regular time intervals or at arbitrary times. Time series data values paired with their corresponding times may be regarded as corresponding to a time-varying function of the time series data.

As used herein, the term “about” refers to an approximately +/−10% variation from a given value. It is to be understood that such a variation is always included in a given value provided herein, whether or not it is specifically referred to.

Unless defined otherwise, all technical and scientific terms used herein have the same meaning as commonly understood by one of ordinary skill in the art to which this invention belongs.

The present invention provides a method and apparatus for obtaining and processing ballistocardiograph data. In accordance with an aspect of the present invention, there is provided an apparatus for determining information indicative of a subject's physiological condition by obtaining and processing ballistocardiograph data. The apparatus comprises a sensor device and a computing device operatively coupled thereto. The sensor device is configured to obtain ballistocardiograph data indicative of heart motion of the subject measured along a plurality of spatial axes. The computing device is configured to receive the ballistocardiograph data from the sensor device and to determine, based on the ballistocardiograph data, processed data indicative of heart motion of the subject. The computing device is further configured to determine one or more indications of the subject's physiological condition based at least in part on the processed data.

In embodiments of the present invention, the apparatus further comprises an output device operatively coupled to the computing device. The output device is configured to provide the one or more indications of the subject's physiological condition. The output device may comprise a video output device such as a computer monitor, sound output system, printer, network communication link, computer memory, or the like. The output device may be configured to provide the one or more indications of the subject's physiological condition for immediate or future viewing by a local or remote user such as a technician, physician, medical staff, or other observer. Indications of the subject's physiological condition may be presented visually along with waveforms indicative of ballistocardiograph data, processed time series data, or the like, or a combination thereof.

In embodiments of the present invention, the apparatus may further comprise an input device configured for receiving operator input directed toward data acquisition operations of the sensor, data processing operations of the computing device, and/or output of physiological conditions. Suitable input devices may comprise a keyboard and mouse, track ball, touch screen, voice recognition system, joystick, portable input device, or the like. For example, the input device may be configured to communicate “start” and “stop” commands to the sensor, thereby starting and stopping collection of ballistocardiograph input data. As another example, the input device may be configured to receive operator input indicative a selection of ballistocardiograph input data to be processed, and/or a manner in which processing is to be performed. For example, operator input may be indicative of time windows or intervals of interest, wherein processing of the ballistocardiograph data comprises processing of ballistocardiograph data indicative of heart motion during said time windows or intervals of interest. An operator may also input quantities to be measured, thereby directing or informing processing operations. This enables an operator to direct processing operations to achieve one or more desired indications of physiological, physical, clinical, or other conditions of interest.

In accordance with another aspect of the present invention, there is provided a method for determining information indicative of a subject's physiological condition by obtaining and processing ballistocardiograph data. The method comprises obtaining ballistocardiograph data indicative of heart motion of the subject measured along a plurality of spatial axes. The method further comprises determining, based on the ballistocardiograph data, processed data indicative of heart motion of the subject. The method further comprises determining one or more indications of the subject's physiological condition based at least in part on the processed data.

In embodiments of the present invention, the method further provides the one or more indications of the subject's physiological condition, for example by presenting the one or more indications via an output device such as a video output device, sound output system, printer, network communication link, storing the one or more indications to computer memory, or the like, or a combination thereof.

In embodiments of the present invention, the method may further comprise receiving operator input directed toward acquisition of ballistocardiograph input data, processing operations related to determination of the processed data, of the computing device, and/or provision of the one or more indications of the subject's physiological condition. For example, operator input may be indicative of when to start and stop collection of ballistocardiograph input data. As another example, operator input may be indicative a selection of ballistocardiograph input data to be processed, and/or a manner in which processing is to be performed. For example, operator input may be indicative of time windows or intervals of interest, wherein processing of the ballistocardiograph data comprises processing of ballistocardiograph data indicative of heart motion during said time windows or intervals of interest. This enables an operator to direct processing operations to achieve one or more desired indications of physiological conditions.

In embodiments of the present invention, the ballistocardiograph data may comprise data collected by a three-axis accelerometer, for example of a sensor device. In some embodiments, the three-axis accelerometer may be configured to collect data indicative of heart motion of the subject along three spatial axes, for example substantially orthogonal x, y, and z-axes are referred to herein. The ballistocardiograph data may thus comprise multidimensional time series data indicative of heart motion of the subject concurrently measured along an x-axis, y-axis and z-axis. For example, the three-axis accelerometer may be configured to output analog or digital data values indicative of acceleration in plural spatial directions due at least in part to and/or correlated with heart motion. The axes may correspond to directions relative to the subject, for example along the direction from head to toe, from the subject's left to right, and from the subject's back to front.

In embodiments of the present invention, determining processed data indicative of heart motion of the subject comprises processing at least a portion of the ballistocardiograph data, or processed data based thereon, to obtain time series data comprising an aggregate representation of the ballistocardiograph data. For example, ballistocardiograph data comprising time series data in three dimensions, such as x, y and z-axis acceleration measurements, may be spatially aggregated into a lower dimensional representation. In some embodiments, aggregation may comprise computing a vector norm of concurrent x-axis, y-axis and z-axis accelerometer measurements, thereby determining a one-dimensional indication of magnitude of heart motion at each of a plurality of times. In embodiments, processing of ballistocardiograph data may comprise determining time series data indicative of one or more physically, medically, clinically, physiologically, or otherwise meaningful quantities related to the subject, such as energy expended or work performed by the heart or rate thereof, timing of cardiac events, cardiac output, blood flow, electrical or neural activity, or the like, or a combination thereof. In some embodiments, each meaningful quantity may be represented as scalar or single-dimensional time series data by aggregation of ballistocardiograph data and/or other data processing operations. Data processing operations may include one or more various operations such as differentiation, averaging, transformation, filtering, or other processing operations related to time series analysis or other analysis or signal processing techniques. Meaningful quantities may be directly observable via the ballistocardiograph data, or indirectly observable but correlated with the ballistocardiograph data, for example through a predetermined model or correlation rule or formula.

In embodiments of the present invention, determining one or more indications of the subject's physiological condition based at least in part on the processed data comprises further aggregation of the processed data, for example to determine one or more values indicative of time series data thereof. For example, processed time series data over a predetermined, selected or operator-defined time window may be time-aggregated by summing, integrating, or averaging the processed data or a function thereof over time to obtain an aggregate value or measurement over said time window. In some embodiments, a thrust summation operation may be invoked to perform such further aggregation. In further embodiments, such further aggregated values may have a meaning such as a physical, physiological, clinical, medical, or diagnostic meaning. This meaning may be interpreted by an operator, technician, physician, or the like, as being indicative of a physiological condition of the subject. In some embodiments, the aggregate value may be an index. In some embodiments, an index may be used in a look-up operation to derive a report related to a physiological condition of the subject.

### Apparatus

Embodiments of the present invention provide an apparatus generally comprising an apparatus for determining information indicative of a subject's physiological condition by obtaining and processing ballistocardiograph data. The apparatus generally comprises a sensor device operatively coupled to a computing device, and optionally operator input and output devices. The sensor device is configured to obtain ballistocardiograph data, for example via a multi-axis accelerometer. The sensor device may further be configured to obtain other data, such as electrocardiograph data, or other data indicative of a subject's physiological condition. The apparatus may typically comprise intermediate electronic components, configured for converting and conveying signals generated by the sensor device to the computing device, for example to provide digital input to the computing device for processing thereby.

In embodiments of the present invention, the apparatus comprises a sensor device and a computing device in a substantially integrated package, such as a portable package, or a collection of packages connected by cables or a wireless interface. The computing device may comprise integral input and/or output devices, or the computing device may comprise a wired or wireless communication interface for coupling with separate input and/or output devices. For example, the computing device in accordance with some embodiments can be a handheld device.

In some embodiments, the apparatus comprises a sensor device separate from but communicatively coupled with a computing device, an input device, and/or an output device via a wired, wireless, direct or networked interface. The sensor device may be compact and easily placed in contact with a subject for obtaining ballistocardiograph data, which is communicated to a separate computing device. In some embodiments, the computing device is nearby and connected by a direct wired or wireless communication interface. In some embodiments, the computing device is communicatively coupled via a wired or wireless interface to a network. In some embodiments in which the computing device is communicatively coupled to a network, the sensor device comprises a network interface for communicating with a network interface device and thereby to the computing device via the network, or the sensor device communicates with the computing device, which in turn communicates with the network.

Various other configurations of the apparatus may be provided, comprising one or more separate or integrated units which are communicatively coupled via direct or network wired or wireless communication to provide for sensor devices, computing devices, and in some embodiments input devices and/or output devices. Communication and control circuitry may be included in each unit to facilitate operative coupling of the units and operation of the apparatus as a whole.

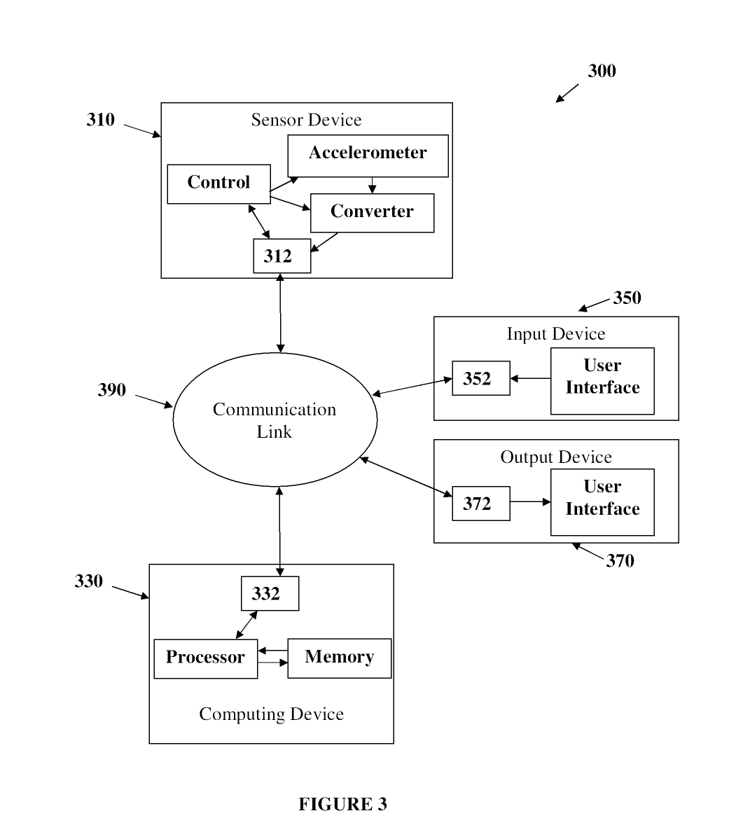

Referring to FIG. 2, an apparatus 100 in accordance with embodiments of the present invention is generally shown. The apparatus 100 includes a sensor device 10 for coupling to a subject and a computing device 12 that is in communication with the sensor device 10. Communication between the sensor and the computing device may be direct or indirect and may include one or more intermediary devices. The communication may be wired or wireless.

The sensor device 10 is provided for detecting, converting and transmitting digital signals corresponding to analog ballistocardiograph (BCG) signals. In some embodiments, as shown in FIG. 2, the sensor device 10 is placed on the sternum of the subject for sensing movement of the chest wall. In some embodiments, other positions suitable for detecting movement of the heart are also contemplated, for example, a transoesophageal sensor such as that described in International Patent Application No. PCT/CA2009/00111 (Publication No. WO2010/015091; herein incorporated by reference in its entirety) could be employed.

In some embodiments, the computing device 12 is provided for receiving the digital signals from the sensor device 10 and analyzing the digital signals. The computing device 12 includes a radio device (not shown), a user interface (not shown), a processor (not shown) and a computer memory (not shown) that stores software that is executable by the processor. The software may alternatively be stored on another type of computer readable medium. The computing device 12 controls the sensor device 10 by sending commands, for example wirelessly via the radio device or by a wired interface, in order to initiate and terminate detection and transmission of the BCG signals. The computing device receives the digital BCG signals and the software is provided to analyze the digital BCG signals received from the sensor and output a report, for example comprising data relating to the BCG signals and/or data relating to the physiological condition of the subject. The report may be, for example, printed by a printer (not shown) that is in communication with the computing device 12 or displayed on a display screen (not shown) of the computing device 12. The report may also be forwarded to and/or saved to local or remote computer memory or database. In some embodiments, for example, the apparatus can be configured for use in a hospital environment and the report may be forwarded to a local area network (LAN) of the hospital where it can be accessed by hospital staff through user stations which communicate through the LAN.

FIG. 3 illustrates an apparatus 300 for determining information indicative of a subject's physiological condition, in accordance with embodiments of the present invention. The apparatus 300 comprises a sensor device 310, a computing device 330, and optionally an input device 350 and/or an output device 370. The sensor device 310, computing device 330, input device 350 and output device 370 are communicatively coupled by communication interfaces 312, 332, 352, and 372, respectively, to each other via a communication link 390. The communication link 390 may comprise a direct wired communication link, a direct wireless communication link, a networked communication link, for example comprising one or more network interfaces and intermediate network components, or a combination thereof. In some embodiments, the communication link 390 may comprise different communication links and/or different types of communication links for linking different sets of devices. In some embodiments, two or more of the sensor device 310, computing device 330, input device 350 and output device 370 may be provided in an integrated package, with the communication link portion therebetween typically being a wired communication link, for example via an internal serial or parallel data connection and/or data bus.

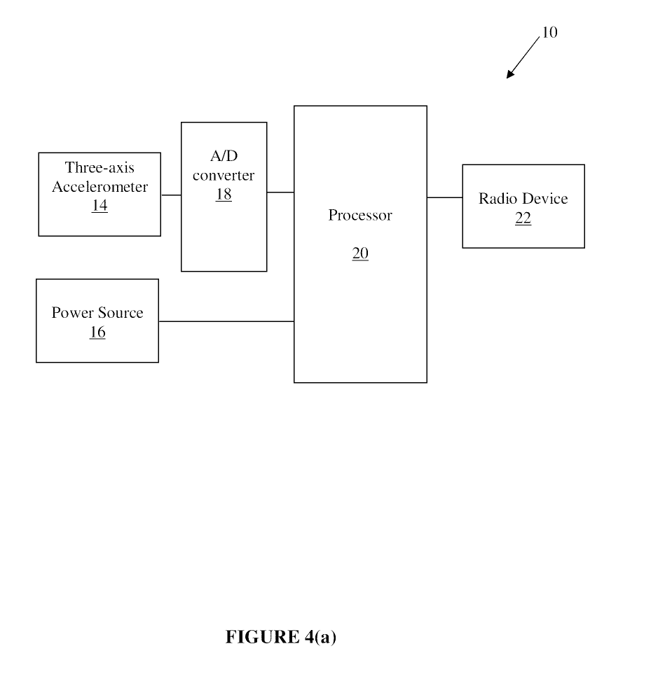

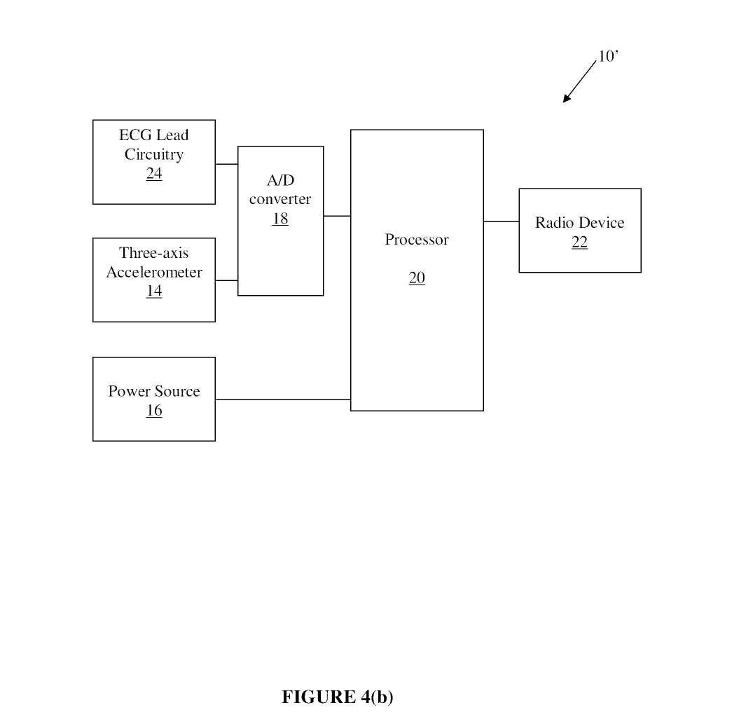

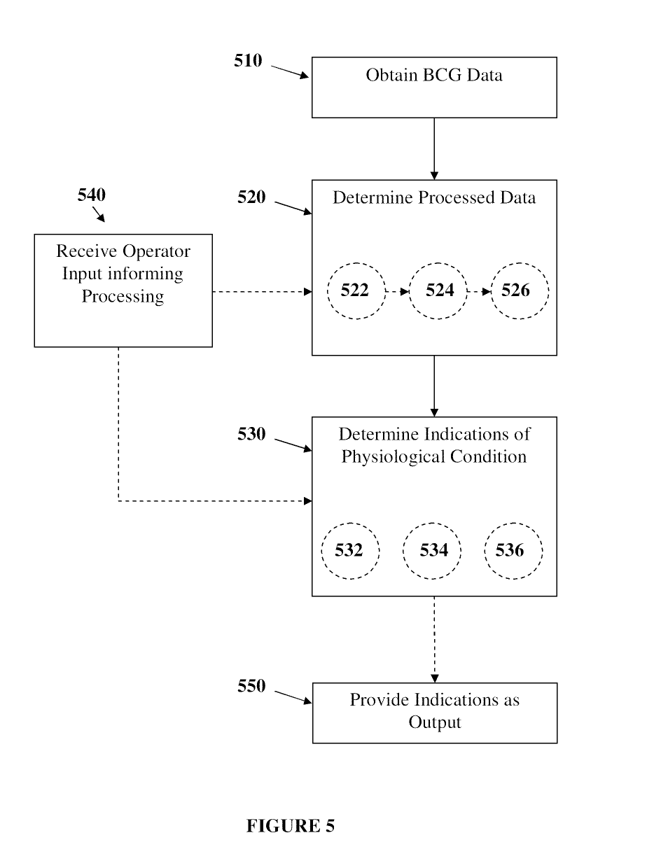

Referring also to FIG. 4(a), in some embodiments the sensor device 10 includes a housing having a contact surface (not shown) for coupling to a subject. The contact surface of the sensor device 10 is provided for coupling to a subject's chest proximal to the sternum. The sensor device 10 includes a three-axis accelerometer 14 for sensing vibrations of a chest wall of the subject. An analog to digital converter 18 is provided in communication with the three-axis accelerometer 14 to receive three separate analog ballistocardiograph signals corresponding to each axis of the three-axis accelerometer 14 and convert the three separate analog signals into digital signals. Analog ballistocardiograph signals may be communicated to separate analog to digital converters, or multiplexed to the same analog to digital converter. Signals output by the one or more analog to digital converters may be parallel or serial, for example.

In addition to the three-axis accelerometer 14 and the analog-to-digital converter 18, the sensor device includes a radio device 22 for transmitting the digital signals to the computing device 12, a processor 20 and a power source 16. In some embodiments, the components of the sensor device 10 are mounted in a housing and provide the sensor device 10 with signal detection, conversion and transmission capabilities. The housing 26 is sized to receive and protect components of the sensor device 10, while still being small enough for mounting on a subject's chest. The housing is made of a biocompatible material such as plastic, for example. The housing may alternatively be made of composite or another suitable material. In alternative embodiments, one or more of the analog-to-digital converter 18, radio device 22 and power source 16 are provided separately to the accelerometer and communicate with the accelerometer via cabling.

In some embodiments, the three-axis accelerometer 14 senses the mechanical motion of the chest wall caused by heart movement in three spatial axes typically denoted x, y and z and outputs three separate BCG signals that correspond to the x, y and z axes. An example of a three-axis accelerometer that is suitable for use in the sensor device 10 is a LIS3L02AL MEMS Inertial sensor, which is manufactured by ST Microelectronics. Alternative embodiments may also be employed, for example multi-axis accelerometers may sense translational or rotational motion such as acceleration and may transmit one or more combined or separate signals indicative thereof. Encoding and communication of analog and/or digital information may be performed in a variety of ways, as would be readily understood by a worker skilled in the art.

In some embodiments, the sensor device 10 further includes a non-volatile memory (not shown) that is programmed with accelerometer calibration data. Calibration of the three-axis accelerometer occurs at the time of manufacture of the sensor device 10 and is typically performed with the aid of a shake table.

The power source 16 is generally a battery capable of providing sufficient power to operate the sensor device 10. The power source 16 may have a finite life, or alternatively, may be rechargeable. The power source 16 may comprise a chemical or electrical energy source, such as a chemical battery, fuel cell, super capacitor, or the like.

In some embodiments, the analog-to-digital converter 18 is provided in communication with the accelerometer 14 to receive three separate analog BCG signals. The BCG signals are amplified by amplifiers set to appropriate gain levels and band-limited by linear filtering prior to being sampled by the analog-to-digital converter 18. Any suitable analog-to-digital converter may be used, such as a 12-bit analog-to-digital converter having a sample rate between 100 samples per second and 2,000,000,000 samples per second, for example between 125 and 1,500 samples per second or between 500 and 1,500,000,000 samples per second. In some embodiments, the sample rate is about 500 samples per second.

The radio device 22 is provided to transmit the digital signals to the computing device 12. Commands for initiating and terminating operation of the sensor device 10 are also transmitted via the radio device 22. The radio device 22 may be any device that is capable of wireless communication. In one embodiment, the radio device 22 is a Bluetooth™ communication device capable of short range wireless communication.

The processor 20 communicates with each of the electronic components of the sensor device 10 and generally controls operation thereof.

In another embodiment, which is shown in FIG. 4(b), sensor device 10′ includes all of the components of sensor device 10 and further includes an electrocardiograph (ECG) sensor for sensing electrical activity of the heart, for example using skin-contacting electrodes.

In some embodiments, the electrocardiograph may comprise (ECG) lead circuitry 24 that is in communication with conductive strips (not shown) that are located at opposite ends of the contact surface. The conductive strips are generally flush with the contact surface and separated so that they are electrically insulated from one another. The conductive strips detect ECG signals through electrode adhesives (not shown), which are provided between the conductive strips and the subject's chest. An example of a sensor device for detecting both ECG and BCG signals is described in PCT Application No. PCT/CA2008/002210, which is herein incorporated by reference in its entirety.

### Obtaining Ballistocardiograph Data

Embodiments of the present invention comprise obtaining ballistocardiograph data indicative of heart motion of the subject measured along a plurality of spatial axes. In some embodiments, at least a portion of the ballistocardiograph data may be obtained using a sensor device comprising a multi-axis accelerometer. A multi-axis accelerometer may be configured to detect and output one or more signals indicative of motion in multiple spatial axes at a single location, for example three substantially orthogonal spatial axes, corresponding to three different directions. An accelerometer may be configured to detect and output a signal indicative of motion, such as magnitude and direction of acceleration, and may be a piezoelectric, piezoresistive, capacitive, MEMS or other type of accelerometer. Accelerometers may be configured or used to detect rotational or translational displacement, vibration, velocity, acceleration, or the like, or a combination thereof.

In embodiments of the present invention, a sensor device is configured to output ballistocardiograph time series data corresponding to one or more streams of accelerometer samples taken in time, for example at a predetermined frequency such as 500 Hz, or at another fixed or variable frequency. The ballistocardiograph time series data may comprise digital data corresponding to accelerometer samples taken in discrete time and taking on quantized sample values.

In some embodiments of the present invention, ballistocardiograph data comprises accelerometer samples taken substantially concurrently along each of a plurality of spatial axes. For example, the ballistocardiograph data may comprise time series data corresponding to streams of x-axis, y-axis and z-axis accelerometer samples, wherein each x-axis sample is taken substantially concurrently with a y-axis sample and a z-axis sample. In some embodiments of the present invention, ballistocardiograph data comprises accelerometer samples taken along each of a plurality of spatial axes, wherein samples along each axis are not taken concurrently but are taken within a predetermined time interval of each other. In some embodiments, such ballistocardiograph data may be processed, for example by interpolation or time shifting, to derive inferred or estimated data indicative of heart motion occurring concurrently along plural spatial axes.

In embodiments of the present invention, ballistocardiograph data may be represented symbolically as a sequence or time series:

v₀, v₁, v₂, . . . ,

comprising data values corresponding to sensor sample times t₀, t₁, t₂, . . . respectively. Together, the sequence of ballistocardiograph data and the sequence of sample times may be used to define a function of ballistocardiograph data versus time. The sequences may be finite, for example each with n elements. In some embodiments, for a given index t, vₜ represents a multidimensional vector of x-axis, y-axis and z-axis accelerometer sample values:

vₜ=(xₜ,yₜ,zₜ).

More or fewer dimensions may also be represented. In some embodiments, for accelerometer samples taken at a frequency f=1/Δt, the sequence of sample times may be represented as: t₀, t₀+Δt, t₀+2Δt, . . . . For example, for accelerometer samples obtained at a frequency of 500 Hz, Δt=2 ms.

In some embodiments, multidimensional ballistocardiograph data may be represented by a plurality of non-concurrent time series data, for example:

x₀, x₁, x₂, . . . ; y₀, y₁, y₂, . . . , z₀, z₁, z₂, . . .

where x-data values taken at sample times t₀, t₁, t₂, . . . ; y-data values taken at possibly different sample times s₀, s₁, s₂, . . . ; and z-data values taken at again possibly different sample times r₀, r₁, r₂, . . . . Linear interpolation, non-linear interpolation, time-shifting, or the like, may be applied to derive inferred or estimated concurrent multidimensional ballistocardiograph data from non-concurrent ballistocardiograph data, as would be readily understood by a worker skilled in the art. Additionally, it will be understood that processing operations described herein with respect to concurrent ballistocardiograph data may similarly be applied directly to non-concurrent ballistocardiograph data without converting it to concurrent ballistocardiograph data.

In some embodiments, the ballistocardiograph data may represent data acquired between a start time and a stop time. In some embodiments, one or more of the start time and the stop time may be determined at least in part by operator input, such as a “start” or “stop” command. In some embodiments, one or more of the start time and the stop time may be determined at least in part by other conditions, such as elapsing of a predetermined amount of time from an operator input or predetermined event, occurrence of a predetermined event, or a combination thereof. For example, the start time may correspond to the time that a “start” command is received, and the stop time may correspond to the time that a “stop” command is received, or the start time plus a predetermined time interval, for example provided by operator input. As another example, the start time may correspond to the time that a “start” command is received plus or minus a predetermined amount of time. If the start time is to be before a “start” command, the apparatus may be configured to acquire and store ballistocardiograph data in a circulating buffer. As is known in the art, a circulating buffer allows for temporary storage of data, which in turn allows for continuous acquisition of data (for example in a standby mode) such that upon receipt of a “start” command, data acquisition can be initiated from a retroactive start time.

As yet another example, the start time may correspond to the time of occurrence of the first cardiac event, of a predetermined type, following a “start” command. Similarly, the stop time may correspond to the time of occurrence of the first cardiac event, of a predetermined type, which occurs after a predetermined time has elapsed from the “start” command, or after a “stop” command, or a combination thereof. You can also say that a

In embodiments of the present invention, the sensor device 10 is coupled to the sternum of the subject using an adhesive and BCG signals are detected thereby. If the sensor device 10′ is used, the sensor device 10′ is adhered to the sternum by electrode adhesives that are used to allow for detection of electrical signals from the heart via the ECG lead circuitry 24. In alternative embodiments, the sensor device may be placed in the esophagus of the subject for sensing vibrations of the wall of the esophagus. When coupled to the chest or positioned in the esophagus, the sensor device may be oriented such that the x-axis of the accelerometer extends in the positive direction from head to toe of a subject, the y-axis of the accelerometer extends in the positive direction from right shoulder to left shoulder of the subject and the z-axis of the accelerometer extends in the positive direction from spine to sternum of the subject. Detection of the signals is initiated for example by a ‘start’ command that is received by the sensor device and detection continues until a stopping condition, for example initiated at least in part by an ‘end’ command is achieved. In one embodiment, the command may be issued by pressing a designated key on an operator input device and/or the computing device that is in communication with the sensor device 10. The same key, or a different key, may then be pressed in order to send a “stop” command to the sensor device upon completion of the test. As the signals are detected, they are amplified and converted to digital signals, for example in real time. Once converted, the digital signals are transmitted to the computing device 12. Once the digital signals are received by the computing device, an analysis of the BCG data is performed and a report relating to the physiological condition of a subject is generated and output by the computing device 12.

### Processing Ballistocardiograph Input Data

Embodiments of the present invention comprise determining, based on the ballistocardiograph data, processed data indicative of heart motion of the subject, and determining one or more indications of the subject's physiological condition based at least in part on said processed data. These operations correspond generally to processing and/or analysis of the ballistocardiograph data, for example by processing said data on a computing device or processor configured in accordance with the present invention.

In embodiments of the present invention, processing of the ballistocardiograph data may comprise a sequence of one or more operations such as filtering, averaging, interpolation, aggregation time-shifting, normalization, quantization, differentiation, integration, multiplication, convolution, correlation, parameterized transformation, or the like, or a combination thereof. Furthermore, it is envisioned that processing of ballistocardiograph time series data may be performed in accordance with operations corresponding to fields such as signal processing, analysis, spectral analysis, calculus, statistical analysis, frequency-domain analysis, wavelet or other transform-based analysis, and the like. Examples of additional analytical tools that may be employed in processing of the ballistocardiograph data include but are not limited to: autocorrelation analysis, cross-correlation analysis, spectral density analysis, cross-spectral density analysis, Fourier analysis, phasor analysis, noise filtering, principal component analysis, singular spectrum analysis, wavelet transform analysis, analysis of rhythmic variance, trend analysis, autoregressive moving average filtering, linear or nonlinear prediction, model-based transformation, pattern recognition, data categorization, feature extraction, trend estimation, system identification, or the like, or a combination thereof.

A general or special purpose computer may be configured to perform analysis, for example via configuration of hardware, software, firmware, or a combination thereof. A computing device may comprise one or more microprocessors operatively coupled to memory and configured to perform numerical processing operations as would be readily understood by a worker skilled in the art.

FIG. 5 illustrates a method for determining information indicative of a subject's physiological condition, in accordance with embodiments of the present invention. The method comprises obtaining ballistocardiograph data 510, for example from a sensor device operatively coupled to a subject and comprising a three-axis accelerometer. The method further comprises determining processed data 520 indicative of heart motion of the subject based on the ballistocardiograph data. The method further comprises determining one or more indications of the subject's physiological condition 530 based at least in part on the processed data. In some embodiments, the method may further comprise receiving operator input 540 informing one or more of determining processed data 520 and determining indications of the subject's physiological condition 530. Operator input may be used for example to determine time intervals of interest for processing, indications of interest to be determined, or the like. In some embodiments, the method may further comprise providing one or more indications of the subject's physiological condition 550, for example via an output device.

In some embodiments, determining processed data 520 indicative of heart motion of the subject may comprise one or more aggregation operations 524, as well as potentially pre-aggregation operations 522 and post-aggregation operations 526, as described herein.

In some embodiments, determining one or more indications of the subject's physiological condition 530 may comprise one or more aggregation operations 532, processing operations 534, and/or index or lookup operations 536, as described herein.

### First Aggregation

In some embodiments, determining processed data may comprise aggregation of processed or unprocessed ballistocardiograph data, such as spatial aggregation. For example, as mentioned above, ballistocardiograph data comprising time series data in three dimensions, such as x, y and z-axis accelerometer measurements, may be spatially aggregated into a lower dimensional representation, such as a one- or two-dimensional representation, for example by computing a vector norm or other lower-dimensional quantity.

For example, time series data represented by the sequence v₀, v₁, v₂, . . . , where each vₜ is a vector representing one or more processed or unprocessed multi-axis accelerometer readings corresponding to a time index t, may be spatially aggregated into processed time series data represented by the sequence:

[symbol] ₀, [symbol] ₁, [symbol] ₂, . . .

by applying a vector magnitude or norm operation:

[symbol]

ₜ=∥vₜ∥.

For example, for vₜ=(xₜ, yₜ, zₜ), the vector magnitude may be computed using the Euclidean norm: [symbol] ₜ=(xₜ²+yₜ²+zₜ²)^{t/2}. It is contemplated that other norms may also be used, such as the L¹ norm, L^{P} norm, or L^{∞} norm. In some embodiments, time series data obtained using such a norm operation may be associated with a “magnitude waveform” of heart motion, since it is indicative of time-evolution of heart motion, measured in magnitude and substantially independent of a particular direction. The aggregate time series [symbol] ₀, [symbol] ₁, [symbol] ₂, . . . may be associated with the same or different times as the vector time series v₀, v₁, v₂, . . . . The aggregate time series may used to define a discrete time-varying function and/or magnitude waveform indicative of an aggregate representation of heart motion.

In some embodiments, an overall heart motion magnitude is determined over a specified time interval. For example, a specified time interval can correspond to a certain portion of the cardiac cycle such as atrial systole, ventricular systole or cardiac diastole, or to an interval between two or more cardiac events, such as depolarization of the inter-ventricular septum (Q), atrial contraction (G), mitral valve close event (H/MVC), isovolumic movement (I), rapid ejection period (J), aortic valve open event (AVO), aortic valve close event (AVC) or mitral valve open event (M/MVO). Other non-limiting examples of useful time intervals are provided in the Examples below.

In embodiments of the present invention, aggregation operations, such as computation of magnitudes of vector-valued time series data, may be used to derive a lower dimensional sequence of values, for example indicative of overall heart motion magnitude independent of a particular direction. Each value in the aggregate sequence may be indicative of a net magnitude of heart motion, force, vibration, or other quantity, obtained for example by taking a norm over vector components, such as spatial x-axis, y-axis and z-axis components. In some embodiments, aggregation operations may be directed at least in part toward producing data having a physical, physiological, clinical, diagnostic, or other meaning to a technician, physician, or the like.

Because the aggregation operation aggregates plural vector components into scalar or a lower-dimensional vector, processing of the BCG data may be performed more quickly and efficiently. Additionally, indications of the subject's physiological condition may be presented in a lower-dimensional space, for example as summary information, which may be more meaningful and easier to understand by a physician or operator. Operator training may also be simplified because annotation of a single waveform may be less subjective to yield more consistent results than annotation based on plural waveforms.

A magnitude waveform or other aggregate waveform may have the further advantage of being robust to rotational motion of a multi-axis accelerometer used in ballistocardiograph data collection. For example, the axis drift due to respiration may be removed by performing a norm operation, since a three-axis accelerometer experiences the same total magnitude of motion regardless of its orientation. Aggregation may thus be used to leverage symmetries and conservation of measured quantities, thereby improving measurement robustness or reliability. This may allow the magnitude waveform amplitudes to be more consistent from beat to beat so that anomalous heart beats can be easily identified. These and other features of the magnitude waveform of BCG data allow it to be useful as an analysis tool to provide relevant information about the physical condition of the heart and the circulatory system.

In some embodiments, aggregation, along with other processing operations, may be directed toward providing physically, medically, clinically, diagnostically, physiologically, or otherwise meaningful quantities related to the subject. Such quantities or an indication thereof may be provided, for example graphically, numerically, audibly, or the like, to a person such as a technician, physician, caregiver, or the like, for use thereby in assessing the subject.

### Pre-Aggregation Processing

In some embodiments, ballistocardiograph data is processed prior to aggregation operations as described above, such as spatial aggregation. For example, ballistocardiograph time series data indicative of multi-axis accelerometer readings may be processed to perform noise filtering, averaging, interpolation, time-shifting, time or value normalization, quantization, aligning of non-synchronous accelerometer readings, or the like. For example, interpolation and/or time shifting may be used to derive concurrent multidimensional ballistiocardiograph data from non-concurrent data.

In some embodiments, pre-aggregation processing may comprise other processing operations or analyses which inherently require non-aggregated data. For example, such processing of sensor device measurements indicative of motion along plural axes may directed toward determinations of: sensor rotation; relative readings along plural measurement axes; vector-based quantities such as divergence, curl, gradient, or Laplacian; spatial or aerial integration; or the like, or a combination thereof.

In some embodiments, pre- or post-aggregation processing may comprise filtering operations, for example to filter out noise, downsample ballistocardiograph data, average ballistocardiograph data, and the like. In some embodiments, ballistocardiograph data may be indicative of plural repetitive heartbeats. In this case, processing may comprise identification of plural sets of data corresponding to separate heartbeats, and processing of the plural sets of data to obtain a representative heartbeat, for example by averaging corresponding data points of each of the plural sets of data.

### Post-Aggregation Processing

In some embodiments, determining processed data from ballistocardiograph data comprises processing data subsequently to one or more aggregation operations. Generally, such post-aggregation processing may comprise of one or more processing operations as described herein, and/or other operations. Post-aggregation processing may be advantageous in that the number of operations required may be reduced due to aggregation. Additionally, post-aggregation quantities may be easier to understand and/or more meaningful to an operator. Aggregation and other processing may also have had the effect of removing extraneous information and/or leveraging symmetries, so that post-aggregation processing may be more effective in determining quantities of interest.

In some embodiments, processing comprises differentiation of aggregate time series data. For example, for time series data represented by the sequence:

[symbol] ₀, [symbol] ₁, [symbol] ₂, . . .

differentiation may be performed by applying a differentiation operation pairwise to the time series data:

d [symbol] ₜ=( [symbol] ₜ₊₁− [symbol] ₜ)/Δt

Here, Δt is the time difference between times t+1 and t, corresponding to time series values [symbol] ₜ₊₁ and [symbol] ₜ, respectively. For example, if the time series data [symbol] ₀, [symbol] ₁, [symbol] ₂ corresponds to a sequence of magnitudes corresponding to ballistocardiograph data samples taken at 500 Hz, then Δt=2 ms. Differentiation may result in time series data denoted by:

d [symbol] ₀, d [symbol] ₁, d [symbol] ₂, . . .

In embodiments wherein the aggregate time series data corresponds to a magnitude waveform of heart motion, differentiation may be regarded as corresponding to a “derivative magnitude” waveform.

In some embodiments, processing may comprise model-based or parameterized processing of data. For example, time series data corresponding to heart motion magnitude may be provided as input into a model-based transformation configured to output time series data corresponding to a correlated quantity, such as ejection fraction. The model-based transformation may comprise a time-domain or frequency-domain transfer function, an autoregressive moving average (ARMA) model, an autoregressive moving average with exogenous inputs (ARMAX) model, or the like. One or more parameters of the model-based transformation may be predetermined, for example in accordance with expert knowledge, parameter estimation due to initialization, operator input, or the like. Such processing of data may be configured for inferring of meaningful information that is correlated, via the model or associated parameters, to heart motion observable from ballistocardiograph data.

In embodiments of the present invention, post-aggregation processing may additionally or alternatively comprise one or more other operations as described herein and/or as would be readily understood. Post-aggregation processing may be directed toward deriving time-series data indicative of aspects of interest, for example related to the subject's physiological condition. In some embodiments, processing operations may correspond to determination of physically meaningful time-dependent quantities such as power, or to determination of physiologically meaningful quantities such as timing of cardiac events, or the like, or a combination thereof.

### Determining Indications of Physiological Condition

Subsequently to determining processed data, such as time series data, indicative of heart motion of the subject, embodiments of the present invention provide for determining one or more indications of the subject's physiological condition based at least in part on the processed data. This may comprise one or more data processing operations, for example implemented by a computing device.

In some embodiments, determining an indication of the subject's physiological condition may comprise time aggregation or other aggregation of time series data into one or more measurements, such as summary measurements, time-independent measurements, indexes related to physiological condition, or the like, or a combination thereof. In some embodiments, an indication of the subject's physiological condition may be implicitly or explicitly associated with units such as physical SI units, physiologically or clinically meaningful units, or the like.

In some embodiments, processing comprises determination of a thrust summation, generally defined as follows. For generic time series data g₀, g₁, g₂, the thrust summation applied on an interval from t=a to t=b is:

[symbol]

=Σ_{t=a . . . b}abs(gₜ₊₁−gₜ).

That is, the thrust summation of a sequence {g} is defined as a sum, over a defined interval, of absolute values of consecutive differences in the sequence {g}.

In some embodiments, the thrust summation may be indicative of changes in value of aggregate time series data such as data corresponding to a derivative magnitude BCG waveform. In some embodiments of the present invention, therefore, ballistocardiograph data corresponding to a stream of three-axis accelerometer samples is processed by taking a norm of vector values to determine processed time series data corresponding to a magnitude waveform, the magnitude waveform is differentiated in time to determine further processed time series data corresponding to a derivative magnitude waveform, and at least a portion of the further processed time series data is processed by applying a thrust summation operation to determine one or more indications of the subject's physiological condition.

The thrust summation may also be useful for calculating indices relating to a physiological condition of a subject. The indices may include: a Systolic Thrust Index, a Systolic Thrust Window, a Recoil Index, a Recoil Window and a Diastolic Ratio, as described herein. Each of the indices may be used to produce a global normal for comparison and/or be used for personal relative comparison purposes.

In embodiments, determining an indication of the subject's physiological condition may comprise other processing operations such as described herein. For example, such operations may correspond to determinations of: power spectral density; goodness of fit of time-series data to a predetermined reference data set; determination of physically meaningful quantities such as total energy, work, or the like; determination of physiologically meaningful quantities such as rhythmic fit relative to a reference or ideal heart operation, cardiac output, ejection fraction, heart operating efficiency, or the like; or a combination thereof. In some embodiments, plural indications of a subject's physiological condition may be presented separately or combined, for example as a ratio such as a diastolic ratio as described herein.

As described herein, determining processed data indicative of heart motion of the subject may comprise one or more operations such as spatial aggregation of time series data, and determining one or more indications of the subject's physiological condition may comprise one or more further operations such as temporal aggregation of the processed time series data. Other processing operations such as those described herein may also be performed in a predefined sequence. It is also contemplated that different operations such as aggregation and/or other processing operations may be applied in different orders, or concurrently, or at least in part in parallel. The order of processing operations as presented herein should not necessarily be considered limiting to the spirit and scope of the invention.

In embodiments of the present invention, operations for determining of processed data and/or indications of the subject's physiological condition may be informed or influenced by operator input. For example, an operator may input a time window or time intervals of interest over which ballistocardiograph data is to be processed, one or more selected indications of the subject's physiological condition to be determined, or the like. An operator may further input annotations corresponding to cardiac events represented by the ballistocardiograph data, such annotations used to inform processing, for example to inform time windows of interest. Annotation may be manual, automated, or semi-automated.

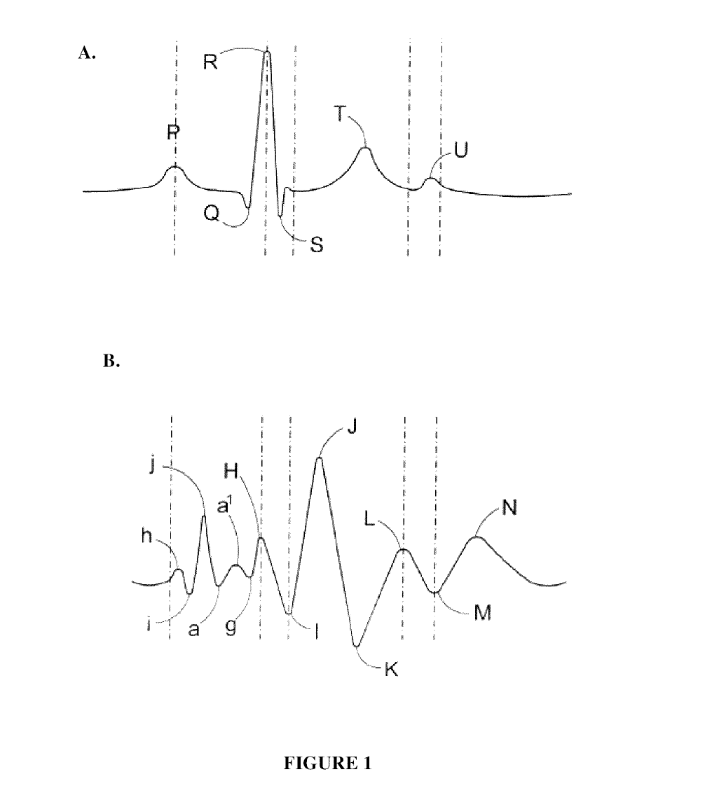

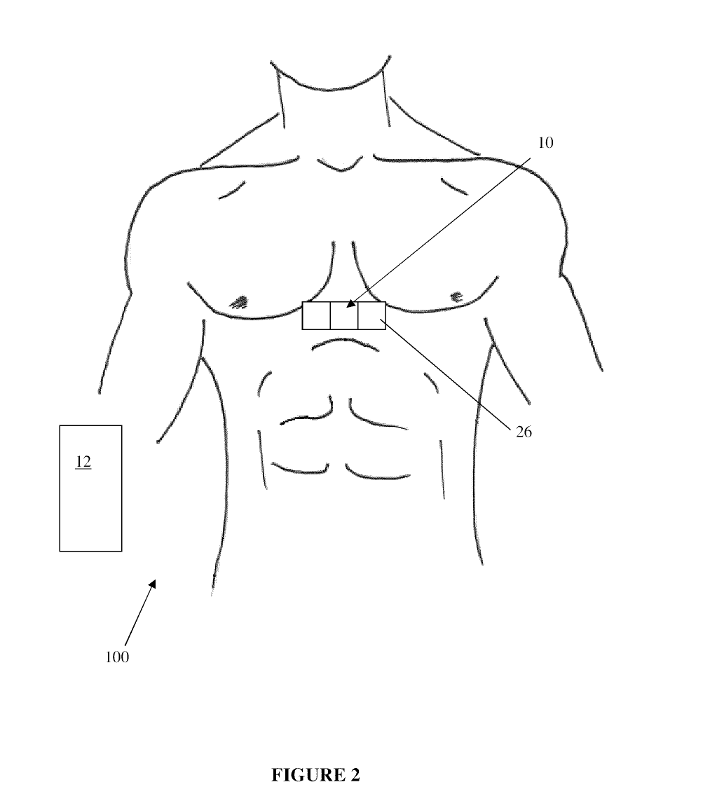

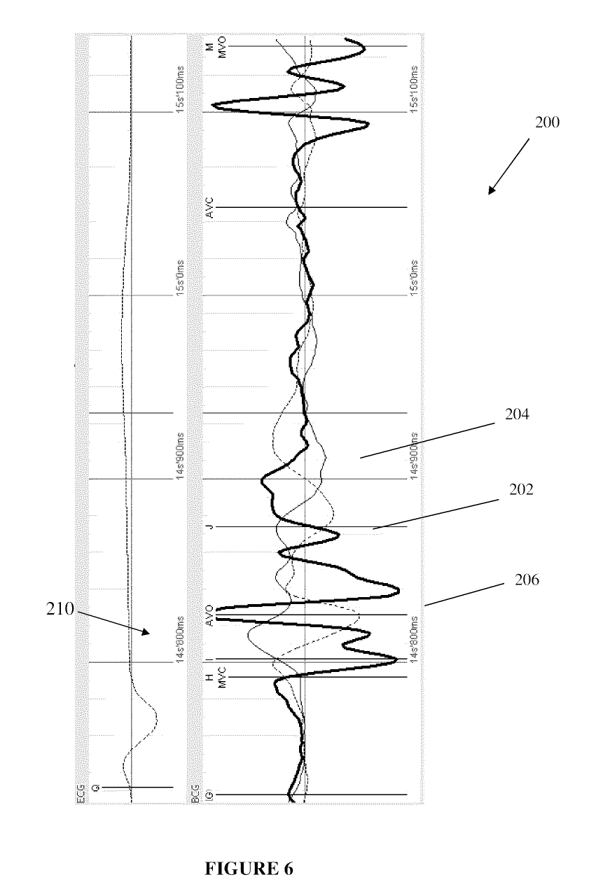

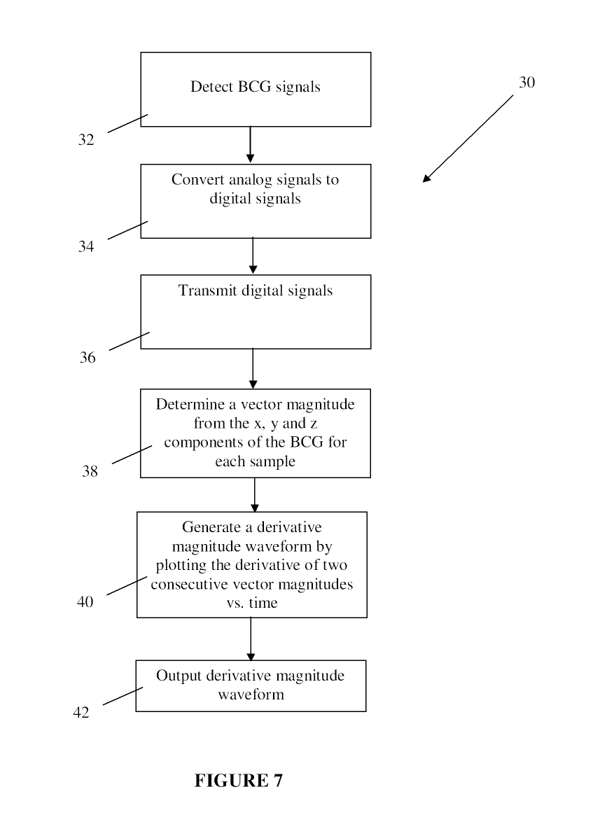

As will be appreciated by a person skilled in the art of electrocardiography and ballistocardiography, the term “annotation” is commonly used to refer to a mark that is provided on a waveform to identify a cardiac event. FIGS. 1(a) and 1(b) show the relationship between arrhythmic electrical functions and related physical motions of a heart in which FIG. 1(a) is a sample ECG waveform and FIG. 1(b) is a sample BCG waveform. Various annotations and methods for marking the annotations on BCG waveforms are described in PCT/CA2008/002209 and PCT/CA2008/002201, which are herein incorporated by reference in their entirety. FIG. 6 depicts an example of a synchronized electrocardiogram and ballistocardiogram waveform pair, on which some of the different cardiac events are identified as described in more detail below.

In some embodiments, operations for determining of processed data and/or indications of the subject's physiological condition may be informed or influenced by automatic recognition of cardiac events. One or more predetermined algorithms or rule sets may be applied to processed or unprocessed ballistocardiograph data to determine cardiac events of interest.

In some embodiments, operations for determining of processed data and/or indications of the subject's physiological condition may be informed or influenced by other data, such as electrocardiograph (ECG) data, or data from one or more other instruments measuring physical or physiological or other conditions of a subject that may be correlated with ballistocardiograph data.

In embodiments of the present invention, one or more indications of the subject's physiological condition may be provided as an index. For example, an index may be a numerical value relative to a predetermined scale. The index value may be looked up, for example in a predetermined look-up table or database, to correlate the index value with information related to the subject's physiological condition, such as full or partial diagnoses, recommended actions, references, or the like. Example indexes as described herein include systolic thrust index, systolic thrust window, recoil index, recoil window, and diastolic ratio. An index may be a global normal index, for example useful in evaluating a subject's condition in context of a population, or a personal relative index, for example useful in evaluating a subject's current condition in context of a series of evaluations of that subject over time.

### Providing Indication of Physiological Condition

Embodiments of the present invention comprise providing the one or more indications of the subject's physiological condition, for example via an output device. The output device may comprise a video output device such as a computer monitor, sound output system, printer, network communication link, local or remote computer memory, or the like, or a combination thereof.

The output device may be configured to provide the one or more indications of the subject's physiological condition for immediate or future viewing by a local or remote user such as a technician, physician, medical staff, or other observer.

Indications of the subject's physiological condition may be presented visually along with waveforms indicative of ballistocardiograph data, processed time series data, or the like, or a combination thereof. Indications of the subject's physiological condition may comprise indexes and/or results of automatic look-up operations based at least in part on such indexes, or the like.

The invention will now be described with reference to specific examples. It will be understood that the following examples are intended to describe embodiments of the invention and are not intended to limit the invention in any way.

### EXAMPLES

### Example 1

The following example relates to specific methods for analyzing ballistocardiograph (BCG) data. An example of a synchronized electrocardiogram-ballistocardiogram (ECG-BCG) waveform set 200 corresponding to BCG data comprising readings from a three-axis accelerometer is shown in FIG. 6. The ECG-BCG waveform set is a visual representation of ECG signal data 210 and BCG signal data 202, 204, 206 that is captured using the sensor device 10′. The ECG-BCG waveform set 200 is automatically synchronized in time because detection of the ECG and BCG signals by the sensor device begins simultaneously in response to the ‘start’ command. As shown, the ballistocardiogram includes three separate waveforms that correspond to the different axes of the accelerometer. The waveforms are identified as follows: the x-axis waveform 202 is shown as a dotted line, the y-axis waveform 204 is shown as a thin line and the z-axis waveform 206 is shown as a thick line. When the sensor device 10 is used, no ECG data is provided.

In order to correlate the ECG and BCG signals detected by the sensor device with heart activity of a subject, each heartbeat of the captured, synchronized ECG-BCG waveform set is annotated with a plurality of different cardiac events. As noted above, the term “annotation” is commonly used to refer to a mark that is provided on a waveform to identify a cardiac event.

FIG. 6 depicts an example of a synchronized electrocardiogram and ballistocardiogram waveform pair, on which some of the different cardiac events are identified using the reference letters: Q, G, H/MVC, I, J, AVO, AVC and M/MVO. The Q annotation denotes depolarization of the inter-ventricular septum; the G annotation denotes atrial contraction; the H annotation denotes the mitral valve close event (MVC); the I annotation denotes isovolumic movement; the J annotation denotes the rapid ejection period; the AVO annotation denotes the aortic valve open event; the AVC annotation denotes the aortic valve close event and the M annotation denotes the mitral valve open event (MVO).

In some embodiments of the present invention, analyzing BCG signals includes using the BCG signals detected by the sensor device 10 to generate a derivative magnitude waveform, as described herein. The derivative magnitude waveform may then be used to provide information about the condition of a heart of a subject.

A method for generating a waveform is generally shown in FIG. 7. The method includes: detecting BCG signals, converting the BCG signals into digital signals and transmitting the digital signals to the computing device 12, as indicated at steps 32, 34 and 36. The BCG signals are detected at a predetermined sample rate. A suitable range of sample rates is 100 Hz to 2 GHz, such as 125 Hz to 1.5 GHz, or 500 Hz to 1.5 GHz. For example, BCG signals may be sampled at 500 Hz, however, higher and lower sample rates are also acceptable. Once the digital signals have been received by the computing device 12, a vector magnitude is determined for each sample, as indicated at step 38.

At step 40, a derivative magnitude function or waveform is generated by plotting a discrete-time derivative or difference in pairs of consecutive vector magnitudes versus time. Time series data corresponding to the derivative magnitude waveform may be computed from time series data corresponding to a magnitude waveform as described herein.

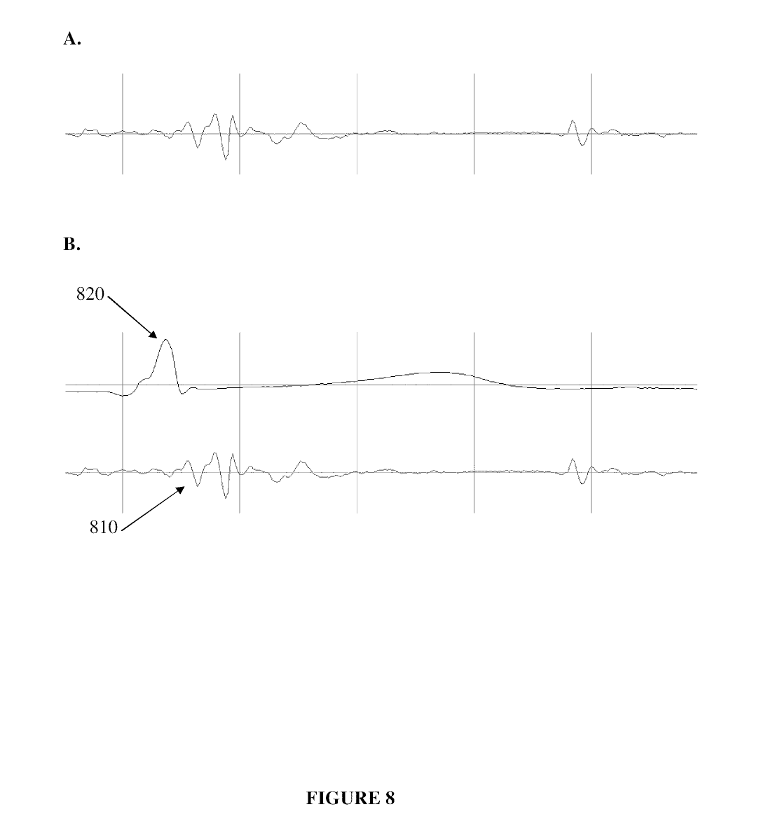

The derivative magnitude waveform plots the derivative of the magnitudes of the vectors calculated from the x, y and z values at the same moment in time. An example of a derivative magnitude waveform obtained using the sensor device 10 is shown in FIG. 8a. An example of a synchronized derivative magnitude waveform 810 paired with an electrocardiogram waveform 820 obtained using the sensor device 10′ is shown in FIG. 8b. As shown, the derivative magnitude waveform is represented as a single line having a repeatable pattern that extends for each heartbeat.

The derivative magnitude waveform is distinctive for each subject and for each test that is performed. Because the derivative magnitude waveform incorporates the x, y and z values from the BCG data into a single waveform, analysis of the BCG data may be performed more quickly. In addition, much of the axis drift from respiration is removed. This allows the waveform amplitudes to be more consistent from beat to beat so that anomalous heart beats can be easily identified. Operator training may also be simplified because annotation of a single waveform may be less subjective to yield more consistent results than annotation based on three waveforms. These and other features of the derivative magnitude waveform of BCG data allow it to be useful as an analysis tool to provide relevant clinical information about the physical condition of the heart and the circulatory system.

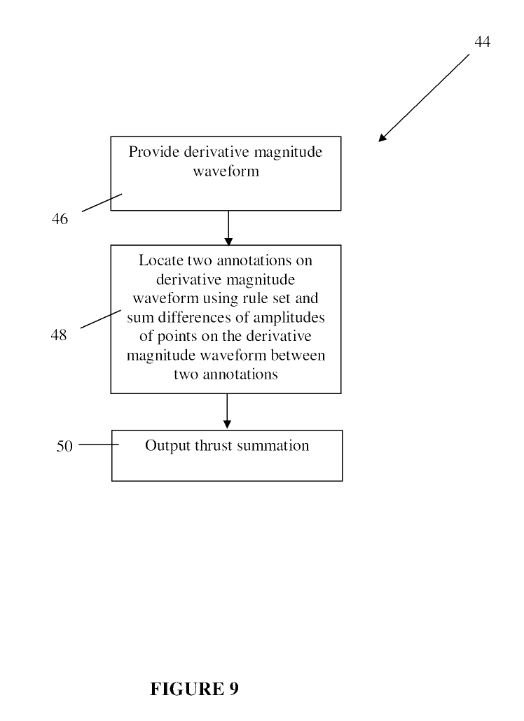

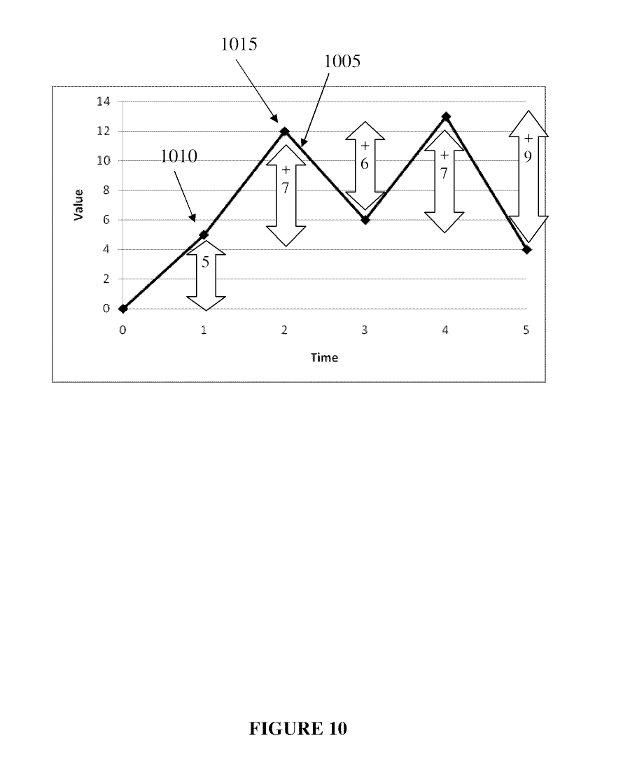

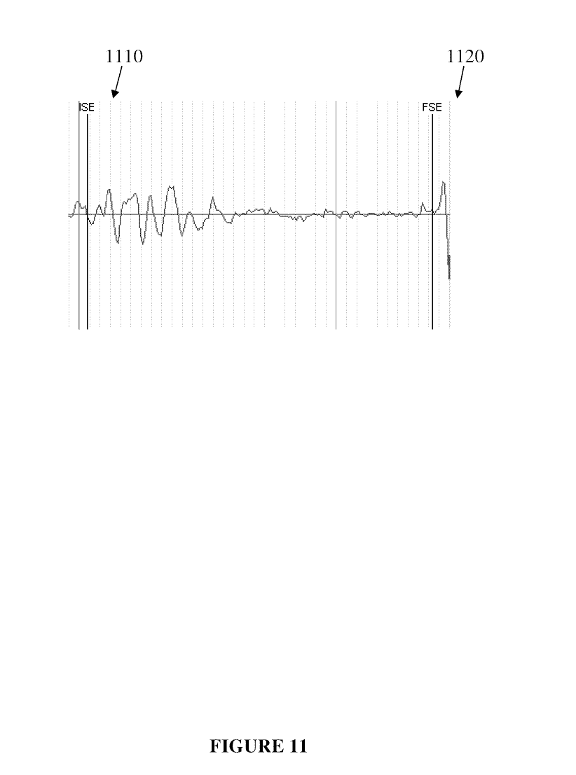

Referring to FIG. 9, a method for analyzing a derivative magnitude waveform 44 is generally shown. The method includes: providing a derivative magnitude waveform, as indicated at step 46, which is generated using the method of FIG. 7, and locating two annotations on the derivative magnitude waveform and summing the absolute differences of amplitudes of subsequent points on the derivative magnitude waveform between two annotations, as indicated at step 48. A thrust summation is then outputted, as indicated at step 50. FIG. 10 depicts the summation of step 48 in which the overall amplitude summation is determined by adding the absolute values of the differences between the amplitudes of subsequent points 1022, on the waveform, as described herein. For example, the height and value of arrow 1005 represents the absolute value of the difference between time series data points 1010 and 1015.

Referring to FIG. 11, a Systolic Thrust Index (STI) is determined on a beat by beat basis between an Initial Systolic Event (ISE) 1110 and a Final Systolic Event (FSE) 1120. The Initial Systolic Event (ISE) is a cardiac event that occurs at the beginning of the forces seen during systole and corresponds to the time that the Mitral Valve Closes (MVC/H). The Final Systolic Event (FSE) is a neutral cardiac event that occurs prior to the forces seen during recoil and corresponds to the time that the Aortic Valve Closes (AVC). The FSE occurs at the end of the T wave on an electrocardiogram. These location parameters are used to provide rules that allow the cardiac events to be located on the derivative magnitude waveform.

The Systolic Thrust Index includes a numerator that is equal to the thrust summation of the derivative magnitude waveform between the ISE and the FSE and a denominator that is the difference in time between the ISE and the FSE.

S [symbol] I= [symbol] _{STI}/ΔT

where [symbol] _{STI} is the thrust summation between the ISE and the FSE, and ΔT is the difference in time between the ISE and the FSE, that is:

[symbol]

_{STI}=Σabs(d [symbol] ᵢ₊₁−d [symbol] ᵢ),

where t_{ISE}≦i<t_{FSE}.

The denominator of the STI is:

ΔT=t_{FSE}−T_{ISE}

The unit for the numerator is milligravities per millisecond (mg/ms). The unit for the denominator is milliseconds (ms).

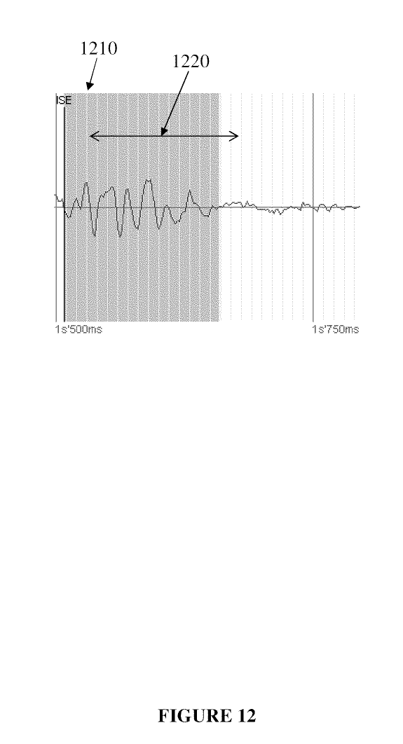

Referring to FIG. 12, the Systolic Thrust Window (STW) is an index calculated on a beat by beat basis from the Initial Systolic Event (ISE) 1210 for a predetermined length of time 1220. The STW is a thrust summation of the derivative magnitude waveform from the ISE for the predetermined length of time. A denominator need not be included because the change in time is constant from beat to beat. A formula for the STW is:

[symbol]

_{STW}=Σabs(d [symbol] ᵢ₊₁−d [symbol] ᵢ)

Where t_{ISE}≦i<t_{ISE+c} and c is a predetermined value.

The unit for the STW is milligravities per millisecond (mg/ms).

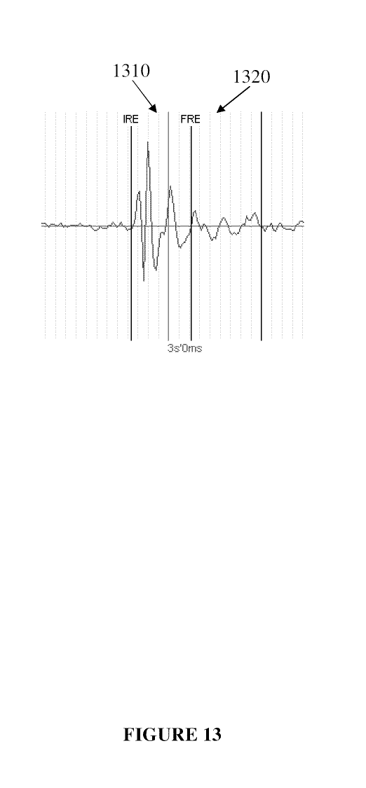

Referring to FIG. 13, the Recoil Index (RI) is determined on a beat by beat basis between an Initial Recoil Event (IRE) 1310 and a Final Recoil Event (FRE) 1320. The Initial Recoil Event (IRE) is a cardiac event that corresponds to the FSE. For example, the Final Recoil Event (FRE) is a cardiac event that may occur, in some embodiments, and in some subjects, approximately 70 to 75 milliseconds following the IRE and may further correspond to the time that the Mitral Valve Opens (MVO). This ensures that all forces that occur as a result of recoil are included. These location parameters are used to provide rules that allow the cardiac events to be located on the derivative magnitude waveform.

The Recoil Index includes a numerator that represents the thrust summation of the derivative magnitude waveform between the IRE and the FRE and a denominator that is the difference in time between the IRE and the FRE.

[symbol]

I= [symbol] _{RI}/ΔT

Where [symbol] _{RI} is the thrust summation between the IRE and the FRE, and ΔT is the difference in time between the IRE and the FRE, that is:

[symbol]

_{RI}=Σabs(d [symbol] ᵢ₊₁−d [symbol] ᵢ)

Where t_{IRE}≦i<t_{FRE}.

The denominator of the RI is:

ΔT=t_{FRE}−t_{IRE}

The unit for the numerator is milligravities per millisecond (mg/ms). The unit for the denominator is milliseconds (ms).

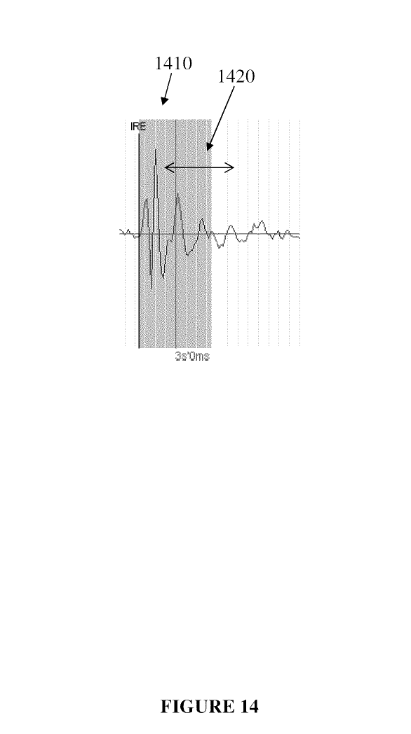

Referring to FIG. 14, the Recoil Window (RW) is determined on a beat by beat basis from the Initial Recoil Event (IRE) 1410 for a predetermined length of time 1420. The RW is a thrust summation of the derivative magnitude waveform from the IRE for a predetermined length of time. A denominator need not be included because the change in time is constant from beat to beat. The formula for the RW is:

[symbol]

_{RW}=Σabs(d [symbol] ᵢ₊₁−d [symbol] ᵢ)

Where t_{IRE}≦i<t_{IRE+c} and c is a constant value.

The unit for the RW is milligravities per millisecond (mg/ms).

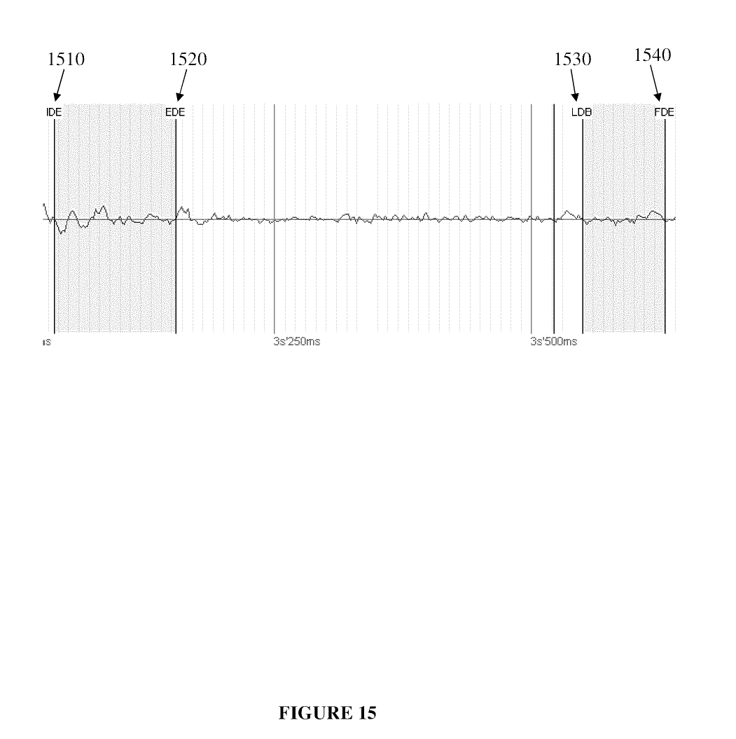

Referring to FIG. 15, the Diastolic Ratio (DR) is determined on a beat by beat basis and is a comparison between two thrust summations. The first thrust summation occurs during Early Diastole (ED) between the Initial Diastolic Event (IDE) 1510 and the Early Diastole End (EDE) 1520. The second thrust summation occurs during Late Diastole (LD) between the Late Diastole Begin (LDB) 1530 and the Final Diastolic Event (FDE) 1540.

In some embodiments, the IDE and the EDE are located on either side of the second positive peak on the z-axis of a three-axis ballistocardiogram following the recoil forces. For example, in some embodiments and subjects, the IDE may be located approximately 50 to 80 ms following the recoil forces and the EDE may be located approximately 100 to 120 ms following the recoil forces. The LDB and FDE are located on either side of the positive peak on the z-axis, which is identified as atrial contraction in the next heart beat. In some embodiments and subjects, the peak may occur approximately 10 to 40 ms prior to the Q wave. These location parameters are used to provide rules that allow the cardiac events to be located on the derivative magnitude waveform, for example by successive approximations. For example, a first rule may be used to estimate a window in which a cardiac event is expected to reside, and a second rule may then be used within the window to locate a predetermined data pattern, such as a peak, valley, flat region, zero crossing, or other pattern, which may be identified with the cardiac event, thereby accurately and effectively determining timing locations of cardiac events based on expert knowledge thereof encoded into embodiments of the present invention.

The DR includes a numerator, which is the thrust summation during ED of the derivative magnitude waveform between the IDE and the EDE, and a denominator, which is the thrust summation during LD of the derivative magnitude waveform between LDB and the FDE. The formula for the DR is:

[symbol]

_{ED}/ [symbol] _{LD}

Where [symbol] _{ED} is the thrust summation during early diastole, and [symbol] _{LD} is the thrust summation during late diastole.

The formula for [symbol] _{ED} can be written as:

[symbol]

_{ED}=Σabs(d [symbol] ᵢ₊₁−d [symbol] ᵢ)

Where t_{IDE}≦i<t_{EDE}.

The formula for [symbol] _{LD} can be written as:

[symbol]

_{LD}=Σabs(d [symbol] ᵢ₊₁−d [symbol] ᵢ)

Where t_{LDE}≦i<t_{FDE}.

Embodiments of the present invention may utilize rules that allow cardiac events of interest to be located relative to processed or unprocessed ballistocardiograph data, for example by successive approximations. For example, a first rule may be used to estimate a window in which a cardiac event of interest is expected to reside, and a second rule may then be used within the window to locate a predetermined data pattern, such as a peak, valley, flat region, zero crossing, or other pattern, which may be identified with the cardiac event, thereby accurately and effectively determining timing locations of cardiac events based on expert knowledge thereof encoded into embodiments of the present invention. Rules may be implemented for determining cardiac events of interest by their relative timing, by absolute characteristics of data indicative of cardiac events, or a combination thereof. Processing of ballistocardiograph data may comprise processing based at least in part on timing of cardiac events of interest, for example by processing time series ballistocardiograph data occurring between two cardiac events of interest, of processing of ballistocardiograph data in a predetermined window before, after, or around a cardiac event of interest.

### Example 2

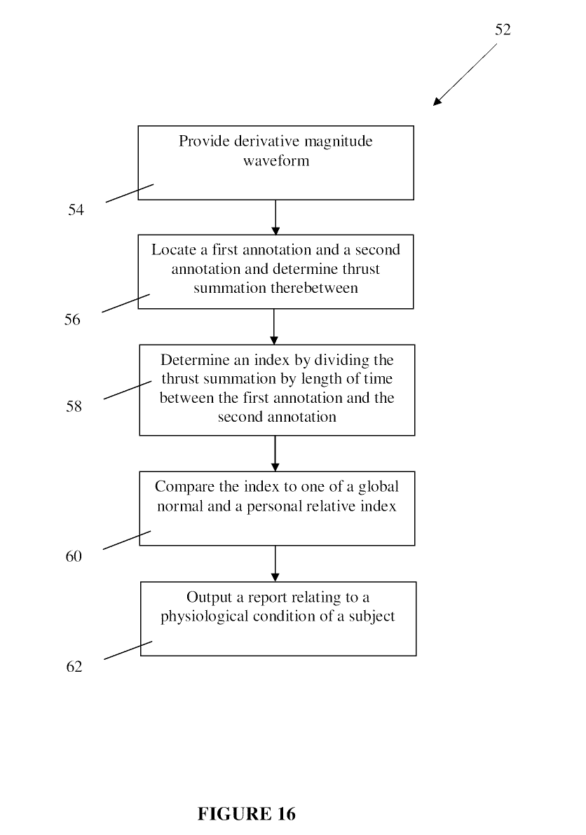

Referring to FIG. 16, a method for determining an index relating to a physiological condition of a subject 52 is shown. At step 54, a derivative magnitude waveform is provided. A thrust summation is then determined between a first annotation and a second annotation, which are located using pre-defined rules, and divided by the length of time between the first annotation and a second annotation to provide the index, as indicated at steps 56 and 58, respectively. The first annotation and the second annotation are located using a rule set that includes rules for locating each cardiac event on the derivative magnitude waveform. At step 60, the index is compared to one of a global normal index or a personal relative index and a report is outputted, as indicated at step 62.

In operation, the sensor device 10 is coupled to the sternum of the subject using an adhesive and BCG signals are detected thereby. When coupled to the chest, the sensor device is oriented such that the x-axis of the accelerometer extends in the positive direction from head to toe of a subject, the y-axis of the accelerometer extends in the positive direction from right shoulder to left shoulder of the subject and the z-axis of the accelerometer extends in the positive direction from spine to sternum of the subject. Detection of the signals is initiated by a ‘start’ command that is received by the sensor device and detection continues until an ‘end’ command is received upon completion of the test. As the signals are detected, they are amplified and converted to digital signals in real time. Once converted, the digital signals are transmitted to the computing device 12. When the digital signals are received by the computing device 12, the x, y and z components of the digital BCG signals are used to generate a derivative magnitude waveform. The derivative magnitude waveform is then searched to locate at least one of the cardiac events: ISE, FSE, IRE, FRE, IDE, EDE, LDB and FDE using a rule set. The rule set includes rules that are structured based on the location parameters that have been previously described with respect to the Systolic Thrust Index (STI), the Systolic Thrust Window (STW), the Recoil Index (RI) and the Diastolic Ratio (DR). Once located, the points corresponding to the at least one cardiac event are stored in computer memory and the selected indices may then be determined using the thrust summation method of FIG. 9. The resulting indices may be compared to global normal indices or, alternatively, compared to a previously determined index for use in a personal relative comparison. A report including the determined indices is then generated and output by the computing device 12.

In one example, a subject's systolic performance is assessed. BCG signals are detected from a subject by performing two tests: i) a pre-exercise test and ii) a post-exercise test. The BCG data that is collected during the pre-exercise test is used as a baseline for comparison with the BCG data that is collected after the subject has completed an exercise, such as 10 deep knee bends, for example. Pre-exercise and post-exercise systolic thrust indices are calculated and the post-exercise systolic thrust index is compared to the pre-exercise systolic thrust index. Differences between the systolic thrust indices can then be evaluated by a physician, or another qualified person, to provide information relating to the subject's systolic performance.

In another example, a subject's diastolic performance is assessed. In this example, pre-exercise and post-exercise tests are performed and the corresponding recoil indices are calculated and compared. Differences between the recoil indices can then be evaluated by a physician, or another qualified person, to provide information relating to the subject's diastolic performance.

In a further example, the value of the Recoil index is utilized for the evaluation and/or monitoring of blood flow volume in a subject. The recoil index can also be used to assess and/or monitor the contractile ability of the heart.

In an additional example, one or more indices of a subject's cardiac performance can be assessed before and after administration of a medication or a course of medication in order to assess and/or monitor the impact of the medication on cardiac performance.

In another example, Systolic Thrust Index and/or the Recoil Index can be used by individuals involved in sporting activities/training as personal comparatives to monitor their cardiac performance.

### Example 3

Other time intervals of interest may be defined for processing of the ballistocardiograph data. For example, ballistocardiograph data in distinct time periods of the cardiac cycle, which are known in the art to be specific to each of the heart valves can be processed for individuals known to suffer from impairment of the function of the valves of the heart and compared to data from individuals with normal valve function. The comparison of data in these specific time windows will aid physicians in the diagnosis and in the measurement of the severity of the valvular dysfunction.

It will be appreciated that, although specific embodiments of the invention have been described herein for purposes of illustration, various modifications may be made without departing from the spirit and scope of the invention. In particular, it is within the scope of the invention to provide a computer program product or program element, or a program storage or memory device such as a solid or fluid transmission medium, magnetic or optical wire, tape or disc, or the like, for storing signals readable by a machine, for controlling the operation of a computer and/or firmware according to the method of the invention and/or to structure its components in accordance with the system of the invention.

In addition, while portions of the above discuss the invention as it can be implemented using a generic OS and/or generic hardware, it is within the scope of the present invention that the method, apparatus and computer program product of the invention can equally be implemented to operate using a non-generic OS and/or can use non-generic hardware.

Further, each step of the method may be executed on any general computer, such as a personal computer, server or the like, or system of computers, and pursuant to one or more, or a part of one or more, program elements, modules or objects generated from any programming language, such as C++, C#, Java, Pl/1, or the like. In addition, each step, or a file or object or the like implementing each said step, may be executed by special purpose hardware or a circuit module designed for that purpose.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

What is claimed is:

1. An apparatus for determining information indicative of a subject's physiological condition, said apparatus comprising:
a) a sensor device configured to obtain ballistocardiograph data indicative of heart motion of the subject measured along a plurality of spatial axes; and
b) a computing device communicatively coupled to the sensor device and configured to receive the ballistocardiograph data therefrom, the computing device configured to determine, based on the ballistocardiograph data, processed data indicative of heart motion of the subject, the computing device further configured to determine one or more indications of the subject's physiological condition based at least in part on the processed data.

2. The apparatus according to claim 1, wherein the sensor device comprises a three-axis accelerometer.

3. The apparatus according to claim 2, wherein the sensor device comprises one or more analog-to-digital converters configured to convert data from the three-axis accelerometer to digital ballistocardiograph data.

4. The apparatus according to claim 1, wherein the ballistocardiograph data comprises time series data.

5. The apparatus according to claim 1, wherein determining said processed data comprises aggregating multidimensional data into a lower dimensional representation.

6. The apparatus according to claim 5, wherein aggregating multidimensional data comprises determining data indicative of a magnitude of the multidimensional data.

7. The apparatus according to claim 1, wherein determining said processed data comprises determining a derivative.

8. The apparatus according to claim 1, wherein determining at least one of said one or more indications of the subject's physiological condition comprises aggregating time series data into one or more measurements thereof.

9. The apparatus according to claim 1, wherein determining at least one of said one or more indications of the subject's physiological condition comprises determining a thrust summation of the processed data.

10. The apparatus according to claim 9, wherein the thrust summation is determined for a portion of the processed data corresponding to time series data between two time points, at least one of the two time points corresponding to a predetermined type of cardiac event.

11. The apparatus according to claim 1, wherein at least one of said one or more indications of the subject's physiological condition is an index.

12. The apparatus according to claim 11, wherein the index is selected from the group consisting of: a systolic thrust index, a systolic thrust window, a recoil index, a recoil window, and a diastolic ratio.

13. The apparatus according to claim 11, wherein the index is a global normal index or a personal relative index.

14. The apparatus according to claim 11, wherein the apparatus is further configured to determine and output a report relating to the subject's physiological condition based at least in part on the index.

15. The apparatus according to claim 1, wherein the one or more indications of the subject's physiological condition relate to one or more aspects of heart operation selected from the group consisting of: work performed, energy expended, rate of work performed, rate of energy expended, timing of heart operations, heart operation efficiency, cardiac output, and ejection fraction.

16. The apparatus according to claim 1, the apparatus further comprising an input device configured for receiving operator input, wherein determining at least one of said processed data and said one or more indications of the subject's physiological condition is based at least in part on said operator input.

17. The apparatus according to claim 16, wherein said operator input comprises input selected from the group consisting of: a time interval of interest, a physiological condition of interest, and an operator-defined annotation corresponding to a cardiac event.

18. The apparatus according to claim 1, wherein determining at least one of said processed data and said one or more indications of the subject's physiological condition is based at least in part on one or more annotations of cardiac events corresponding to the ballistocardiograph data.

19. The apparatus according to claim 18, wherein the one or more annotations are based at least in part on operator input.

20. The apparatus according to claim 19, wherein the one or more annotations are determined at least in part automatically.

21. The apparatus according to claim 1, the apparatus further comprising an output device operatively coupled to the computing device, the output device configured to provide the one or more indications of the subject's physiological condition.

22. The apparatus according to claim 21, wherein the output device is configured to present a visual representation of the ballistocardiograph data along with at least one of the indications of the subject's physiological condition.

23. The apparatus according to claim 1, wherein the processed data comprises time series data indicative of derivative magnitude of heart motion, and wherein the one or more indications of the subject's physiological condition comprises a thrust summation corresponding to a predetermined portion of said time series data.

24. A method for determining information indicative of a subject's physiological condition, said method comprising:
a) obtaining ballistocardiograph data indicative of heart motion of the subject measured along a plurality of spatial axes;
b) determining, based on the ballistocardiograph data, processed data indicative of heart motion of the subject; and
c) determining one or more indications of the subject's physiological condition based at least in part on the processed data.

25. The method according to claim 24, wherein the ballistocardiograph data is indicative of heart motion of the subject measured along three-axes.

26. The method according to claim 24, wherein the ballistocardiograph data comprises time series data.

27. The method according to claim 24, wherein determining said processed data comprises aggregating multidimensional data into a lower dimensional representation.

28. The method according to claim 27, wherein aggregating multidimensional data comprises determining data indicative of a magnitude of the multidimensional data.

29. The method according to claim 24, wherein determining said processed data comprises determining a derivative.

30. The method according to claim 24, wherein determining at least one of said one or more indications of the subject's physiological condition comprises aggregating time series data into one or more measurements thereof.

31. The method according to claim 24, wherein determining at least one of said one or more indications of the subject's physiological condition comprises determining a thrust summation of the processed data.

32. The method according to claim 31, wherein the thrust summation is determined for a portion of the processed data corresponding to time series data between two time points, at least one of the two time points corresponding to a predetermined type of cardiac event.

33. The method according to claim 24, wherein at least one of said one or more indications of the subject's physiological condition is an index.

34. The method according to claim 33, wherein the index is selected from the group consisting of: a systolic thrust index, a systolic thrust window, a recoil index, a recoil window, and a diastolic ratio.

35. The method according to claim 33, wherein the index is a global normal index or a personal relative index.

36. The method according to claim 24, wherein the one or more indications of the subject's physiological condition relate to one or more aspects of heart operation selected from the group consisting of: work performed, energy expended, rate of work performed, rate of energy expended, timing of heart operations, heart operation efficiency, cardiac output, and ejection fraction.

37. The method according to claim 24, the method further comprising receiving operator input, wherein determining at least one of said processed data and said one or more indications of the subject's physiological condition is based at least in part on said operator input.

38. The method according to claim 37, wherein said operator input comprises input selected from the group consisting of: a time interval of interest, a physiological condition of interest, and an operator-defined annotation corresponding to a cardiac event.

39. The method according to claim 24, wherein determining at least one of said processed data and said one or more indications of the subject's physiological condition is based at least in part on one or more annotations of cardiac events corresponding to the ballistocardiograph data.

40. The method according to claim 39, wherein the one or more annotations are based at least in part on operator input.

41. The method according to claim 39, wherein the one or more annotations are determined at least in part automatically.

42. The method according to claim 24, the method further comprising communicating or storing the one or more indications of the subject's physiological condition.

43. The method according to claim 24, the method further comprising presenting a visual representation of the ballistocardiograph data along with at least one of the indications of the subject's physiological condition.

44. The method according to claim 24, wherein the processed data comprises time series data indicative of derivative magnitude of heart motion, and wherein the one or more indications of the subject's physiological condition comprises a thrust summation corresponding to a predetermined portion of said time series data.

45. A computer program product comprising a memory having computer readable code embodied therein, for execution by a CPU, for performing a method for determining information indicative of a subject's physiological condition based on ballistocardiograph data indicative of heart motion of the subject measured along a plurality of spatial axes, said method comprising:
a) determining, based on the ballistocardiograph data, processed data indicative of heart motion of the subject; and
b) determining one or more indications of the subject's physiological condition based at least in part on the processed data.
